(19) United States
(12) Patent Application Publication
(10) Pub. No.: US 2026/0017562 A1
(43) Pub. Date: Jan. 15, 2026

(54) Concept Mapping Using Fine-Tuned Large Language Models

(71) Applicant: Cerner Innovation, Inc., Kansas City, MO (US)

(72) Inventors: Pragnya Ranjan Pradhan, Similipada (IN); Suman Pal, Bangalore (IN); Oshin Benny Anto, Kerala (IN); Rupanjali Chaudhuri, Bangalore (IN)

(73) Assignee: Cerner Innovation, Inc., Kansas City, MO (US)

(21) Appl. No.: 18/919,162

(22) Filed: Oct. 17, 2024

Related U.S. Application Data
(60) Provisional application No. 63/670,356, filed on Jul. 12, 2024.

(51) Int. Cl. G06N 20/00; G16H 10/60

(52) U.S. Cl. CPC G06N 20/00; G16H 10/60

(57) ABSTRACT

Techniques for fine-tuning a pre-trained vector embedding model for recommending standard codes for mapping with proprietary codes are disclosed. Proprietary codes, as referred to herein, include reference codes particular to organizations or vendors. Standard codes, as referred to herein, are industry or standardized codes. The system access a candidate set of standard codes that have been mapped to one or more proprietary codes. The system determines a number of times a standard code is mapped to a proprietary code. Standard codes that have been mapped to proprietary codes a number of times that meet a threshold are selected to be included in a training set for fine-tuning the pre-trained vector embedding model. Standard codes with a number of mappings that does not meet the threshold are not included in the training set. The system may also use vector embedding models for generating aggregated datasets for datasets of the training set.

### BENEFIT CLAIMS; RELATED APPLICATIONS; INCORPORATION BY REFERENCE

This application claims the benefit of U.S. Provisional Patent Application 63/670,356, filed Jul. 12, 2024, and is hereby incorporated by reference.

The Applicant hereby rescinds any disclaimer of claim scope in the parent application(s) or the prosecution history thereof and advises the USPTO that the claims in this application may be broader than any claim in the parent application.

U.S. patent application Ser. No. 18/410,219 titled, “Concept Mapping Using Large Language Models,” filed on Jan. 16, 2024, (Attorney Docket No. R01224NP) is hereby incorporated by reference.

### TECHNICAL FIELD

The present disclosure relates to concept mapping using large language models. In particular, the present disclosure relates to fine-tuning large language models for use in concept mapping.

### BACKGROUND

Electronic health records (EHRs) are commonly stored in diverse formats and encoded with institution-specific concepts. Different formats and institution-specific concepts lead to ambiguity in local/client specific coding systems. The ambiguity stems from various factors, including client specific developed acronyms and synonyms used by laboratories as well as errors, such as misspellings and omissions in manual data entry. The variability in data encoding poses a significant challenge to multi-site clinical information exchange.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1A illustrates a system for concept mapping in accordance with one or more embodiments;

FIG. 1B illustrates a system for fine-tuning a vector embedding model in accordance with one or more embodiments;

FIG. 2 illustrates an example set of operations for generating a training set for use in fine-tuning a pretrained vector embedding model in accordance with one or more embodiments;

FIG. 3 illustrates example datasets of a training set for fine-tuning a pre-trained vector embedding model;

FIG. 4 illustrates an example set of parameters for fine-tuning a pre-trained vector embedding model;

FIG. 5 illustrate a comparison between a pre-trained vector embedding model and a fine-tuned vector embedding model; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. EVENT CODE MAPPING SYSTEM ARCHITECTURE
3. FINE-TUNING SYSTEM ARCHITECTURE
4. GENERATING DATASETS FOR FINE-TUNING PRE-TRAINED VECTOR EMBEDDING MODELS
5. EXAMPLE DATASETS OF A TRAINING SET FOR FINE-TUNING PRE-TRAINED VECTOR EMBEDDING MODELS
6. EXAMPLE PARAMETERS FOR FINE-TUNING PRE-TRAINED VECTOR EMBEDDING MODELS
7. PERFORMANCE COMPARISON OF PRE-TRAINED VECTOR EMBEDDING MODEL AND FINE-TUNED VECTOR EMBEDDING MODEL
8. PRACTICAL APPLICATION; IMPROVEMENTS & ADVANTAGES
9. HARDWARE OVERVIEW
10. MISCELLANEOUS; EXTENSIONS

### 1. General Overview

One or more embodiments include fine-tuning a pre-trained vector embedding model, e.g., SAPBERT, BIOBERT, BIO-CLINICALBERT, for recommending standard codes for mapping with proprietary codes. Proprietary codes, as referred to herein, include reference codes particular to organizations or vendors. Standard codes, as referred to herein, are industry or standardized codes, e.g., LOINC, SNOMED-CT, RxNorm. Mapping proprietary codes to standard codes enhances data interoperability and plays a crucial role in improving the overall quality of healthcare delivery and patient outcomes.

Initially, the system access a candidate set of standard codes that have been mapped to proprietary codes. The system determines a number of times a standard code is mapped to a proprietary code. Standard codes that have been mapped to proprietary codes a number of times that meet a threshold are selected to be included in a training set for fine-tuning the pre-trained vector embedding model. Standard codes with a number that does not meet the threshold are not included in the training set.

One or more embodiments generate aggregated datasets corresponding to the selected standard codes. First vector embeddings are generated for first datasets of the respective standard codes. Second vector embeddings are generated for second datasets of the respective standard codes. The system computes a similarity measure for the first vector embeddings and the second vector embeddings of the respective standard codes. When a similarity measure meets a threshold measure, the second dataset representing the respective standard code is included in the aggregated dataset for the respective standard code. When the similarity measure does not meet the threshold measure, the second dataset representing the respective standard code is not included in the aggregated dataset representing the respective standard code.

One or more embodiments generate a training set that comprises (a) an identifier or label corresponding to a standard code, (b) an aggregated dataset corresponding to the standard code, and (c) an aggregated dataset corresponding to the one or more datasets of the proprietary code that has been mapped to the standard code. The aggregated datasets may be pre-processed prior to applying the pre-trained vector embedding model to the training set.

One or more embodiments described in this Specification and/or recited in the claims may not be included in this General Overview section.

### 2. Event Code Mapping System Architecture

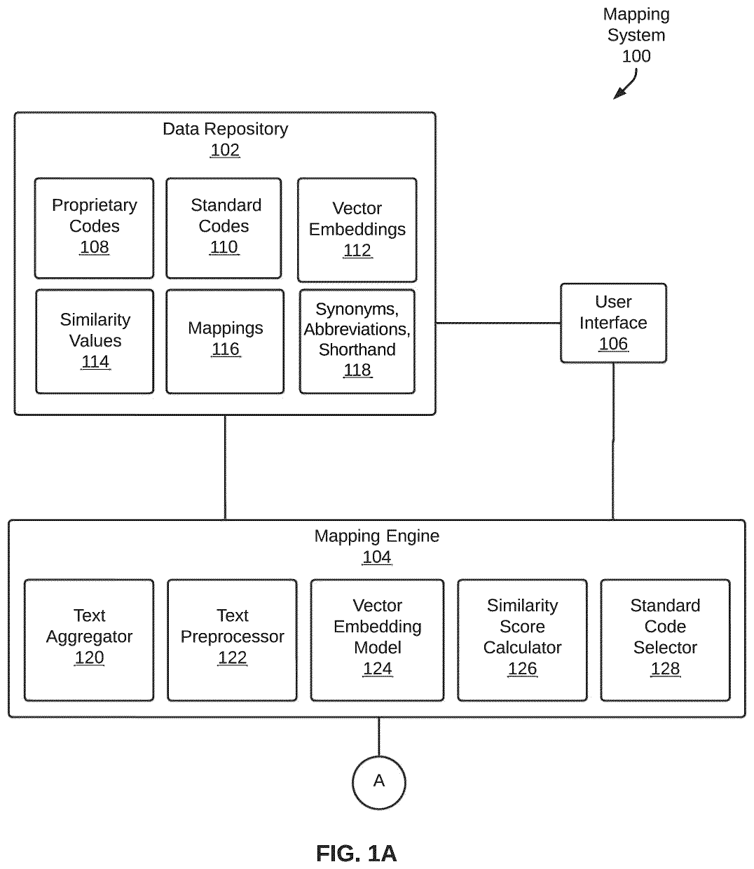

FIG. 1A illustrates a mapping system 100 in accordance with one or more embodiments. As illustrated in FIG. 1A, the system 100 includes a data repository 102, a mapping engine 104, and a user interface 106. In one or more embodiments, the system 100 may include more or fewer components than the components illustrated in FIG. 1A. The components illustrated in FIG. 1A may be local to or remote from each other. The components illustrated in FIG. 1A may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Furthermore, a data repository 102 may include multiple different storage units and/or devices. The multiple different storage units and/or devices may or may not be of the same type or located at the same physical site. Furthermore, a data repository 102 may be implemented or executed on the same computing system as the mapping engine 104 and the user interface 106. Additionally, or alternatively, a data repository 102 may be implemented or executed on a computing system separate from the mapping engine 104 and the user interface 106. The data repository 102 may be communicatively coupled to the mapping engine 104 and the user interface 106 via a direct connection or via a network.

In one or more embodiments, the data repository 102 is populated with information from a variety of sources and/or systems. The data repository 102 may be populated with data, such as proprietary codes 108, standard codes 110, vector embeddings 112, similarity values 114, mappings 116, and synonyms, abbreviations, and shorthand 118. Any of this information may be stored in a structured format (e.g., a table).

In one or more embodiments, proprietary codes 108 are reference codes for clinical and/or non-clinical events that are customized for consumers. When creating proprietary codes 108, local practice may be favored over uniformity of content, resulting in different consumers having unique sets of proprietary codes 108. Although the names of the proprietary codes 108 may differ between consumers, many of the proprietary codes 108 have semantic equivalences. Mapped proprietary codes are proprietary codes that have been mapped to a standard code, e.g., LOINC, SNOMED-CT, RXNorm. Unmapped proprietary codes are codes that have not been mapped to a standard code.

In embodiments, proprietary codes 108 include attributes or variables, i.e., reference data, for identifying clinical and/or non-clinical events. The proprietary codes 108, mapped and unmapped, may be sourced from one or more disparate consumer databases. The attributes for each of the proprietary codes 108 may be sorted into groups, e.g., a “Names” attribute group and an “Extras” attribute group. The “Names” attribute group may include consumer specific codes, descriptions, identifies, and/or unit measurement types. For example, the “Names” attribute group may include Code Name, Code Alternate Name, DTA (Discrete Task Assay), and Specimen. The “Extras” attribute group may include an event set hierarchy and/or additional reference data. An event set hierarchy is a hierarchical or parent/child relationship of events sets. The additional reference data may include a co-occurring unit. Co-occurring units are associated units to the value for the event code data collected.

In some embodiments, the proprietary codes 108 include Code Set 72. Code Set 72, also known as Cerner Clinical Event Codes, is a proprietary code set maintained by Cerner Corporation. Code Set 72 is an extensive collection of codes used to represent various clinical and non-clinical events, including clinical documents, note types, immunizations, and clinical observations, such as laboratory results and vital signs. Code Set 72 is highly customized by Cerner clients, and the specific codes used may vary depending on the client's healthcare system. The general structure and purpose of the code set remain consistent across Cerner clients. Code Set 72 is a very large code set, encompassing a wide range of clinical events. The specific codes used in Code Set 72 are tailored to meet the specific needs of each Cerner client.

In one or more embodiments, the standard codes 110 are sets of industry or standardized codes that are widely adopted and used across the healthcare industry. Standard codes 110 represent various aspects of patient care, procedures, diagnoses, and other healthcare-related information. Example standard codes include International Classification of Diseases, 10^{th} Edition (ICD-10), Current Procedural Terminology (CPT), Healthcare Common Procedure Coding System (HCPCS), Systematized Nomenclature of Medicine Clinical Terms (SNOMED-CT), National Drug Code (NDC), and RxNorm. A standard code may be mapped to multiple proprietary codes.

In embodiments, the standard codes 110 are Logical Observation Identifiers Names and Codes (LOINC®). LOINC is a universal standard for identifying health measurements, observations, and documents. LOINC is a common language that allows different healthcare systems to exchange data seamlessly. LOINC codes are used to represent the “question” for a test or measurement, such as “blood glucose” or “body mass index,” to aid in ensuring that the results of tests and measurements are interpreted accurately and consistently across different systems. The LOINC database contains over 90,000 codes that are translated into more than 40 languages. LOINC is used by a wide variety of organizations, including hospitals, clinics, laboratories, and government agencies. LOINC helps to ensure that data can be exchanged seamlessly between different healthcare systems, thereby improving patient care by making it easier for clinicians to access and understand patient data. LOINC codes are unique and unambiguous; this helps to reduce errors in data entry and interpretation. LOINC can be used to link data from different sources, improving research on a variety of health topics.

In embodiments, standard codes 110 include attributes or variables, i.e., reference data, for identifying clinical and/or non-clinical events. Similar to the proprietary codes 108, attributes for each of the standard codes 110 may be sorted into groups. A “Names” attribute group may include code names, code references, and/or observations. For example, the “Names” attribute group for a LOINC code includes Long Common Name, Short Name, Related Names 2, and Six axes of LOINC. Long Common Names are designed to be the user-friendly representation of a LOINC term, providing a human-readable format for understanding the meaning of a LOINC code. The Related Names 2 are synonyms that are associated with the specific LOINC code.

The Six axes of LOINC include component, property, time, system, scale, and method. The component axis represents the analyte or property being measured. The component axis describes what is being observed or measured, such as glucose, cholesterol, or blood pressure. The property axis describes the characteristics of the analyte or property. The property axis provides additional information about the type of measurement being made, such as mass, concentration, or time. The time axis specifies the timing of the observation, indicating when the measurement was taken or how the observation is related to time. For example, the time axis might indicate if the observation is a point in time, a 24-hour urine collection, or a fasting specimen. The system axis specifies the system or specimen source from where the observation is derived. The system axis provides information about the origin of the specimen, such as blood, urine, or cerebrospinal fluid. The scale axis describes the scale of measurement for the observation, such as qualitative, ordinal, or quantitative. The scale axis provides information about how the observation is expressed numerically or categorically. The method axis represents the procedure or method used to perform the observation. The method axis provides details about the specific technique, instrument, or protocol used to obtain the result.

In one or more embodiments, the vector embeddings 112 in the data repository 102 are text that have been converted to a numeric format. The vector embeddings 112 are representations of individual words for text analysis, typically in the form of a real-valued vector. The vector embeddings 112 may represent individual text or may represent an aggregation of text. As will be described in further detail below with respect to mapping engine 104, the vector embeddings 112 may be formed using various word embedding techniques. The vector embeddings 112 represent mapped and unmapped standard codes and unmapped proprietary codes. The vector embeddings 112 may also represent datasets for different attributes of respective standard codes and/or respective proprietary codes.

In one or more embodiments, the similarity values or measures 114 in the data repository 102 indicate the similarity between vector embeddings. The similarity values 114 may be of vector embeddings of datasets for mapped or unmapped standard code as well as unmapped proprietary codes. The higher the similarity values 114, i.e., the closer to 1.0, the greater a semantic match between vector embeddings. The similarity values 114 may each be assigned a ranking category. For example, a similarity value less than 0.90 may be categorized as “low”; a similarity value equal to or greater than 0.90 and less than 0.98 may be categorized as “medium”; and a similarity value greater than or equal to 0.98 may be categorized as “high”. The similarity values 114 may be weighted to reflect the relevance of the type of data used to calculate the vector embeddings. For example, data with a high relevance to determining an appropriate mapping of a proprietary code may receive a weight of 0.55, while data with less relevance to the mapping may receive a weight of 0.45.

In one or more embodiments, mappings 116 include mappings between proprietary codes 108 and standard codes 110. When a mapped standard code is mapped to an unmapped proprietary code, the unmapped proprietary code provides a dataset for the mapped standard code that may be used for future charting. When an unmapped standard code is mapped to a proprietary code 108, the unmapped standard code becomes a mapped standard code. Multiple proprietary codes may be mapped to a standard code.

In one or more embodiments, the synonyms, abbreviations, and shorthand 118 are included in a table that provides synonyms, abbreviations, and/or shorthand that may or may not be specific to a consumer and corresponding expansions for the respective synonym, abbreviation, or shorthand. For example, “SBP” may correspond to “systolic blood pressure”; “LMP” may correspond to “last menstrual period”; “I:E” may correspond to “inspiratory to expiratory ratio”; and “GAD7” may correspond to “general anxiety disorder”.

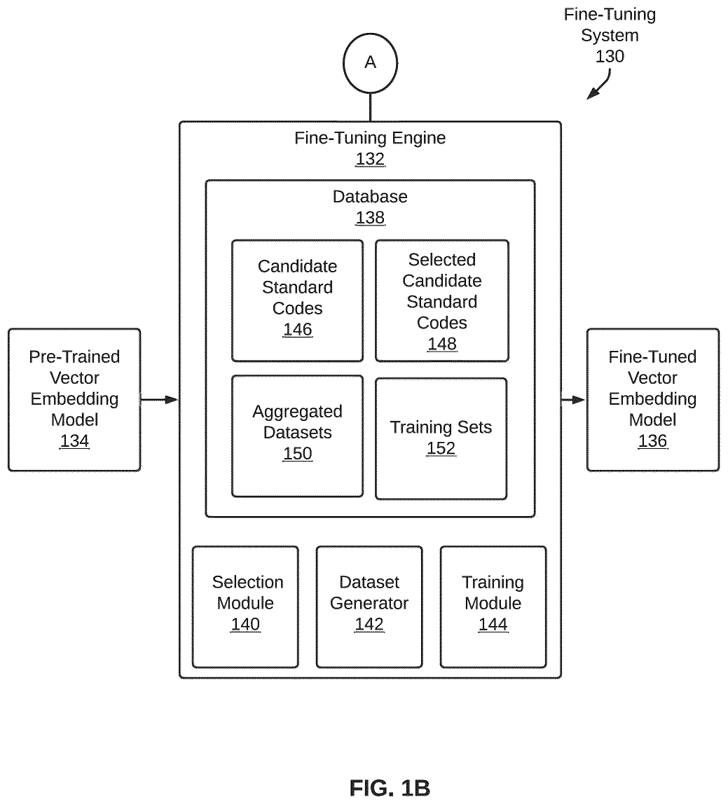

In one or more embodiments, the mapping engine 104 of the system 100 is hardware and/or software configured to map unmapped proprietary codes to mapped and unmapped standard codes. Examples of operations for providing recommendations of candidate mapped and unmapped standard codes are described below with references to FIGS. 2A-2C. The mapping engine 104 may include a text aggregator 120, a text preprocessor 122, a vector embedding model 124, a similarity score calculator 126, and a standard code selector 128.

In one or more embodiments, the text aggregator 120 aggregates text from the attributes of the proprietary codes 108 and the attributes of the standard codes 110. The text aggregator 120 may aggregate text prior to, or after, preprocessing of the text by the text preprocessor 122.

In some embodiments, the text is processed by the text preprocessor 122 prior to applying the vector embedding model 124 to the aggregated text to generate vector embeddings 112. The text preprocessor may perform functions, such as converting the text into lower case and/or retaining numeric tokens. Text is converted to lower case to provide uniformity to the text. In prior art mapping engines, numeric tokens are typically removed during text preprocessing. Removal of numeric tokens may eliminate a distinguishing feature of a concept. For example, “Right Ear 500 Hz POC” and “Right Ear 1000 Hz POC” are differentiated using a numeric token. By retaining numeric tokens, misclassifications are more readily avoided.

In embodiments, text preprocessing may further include handling special characters, removing unwanted text, and customizing preprocessing. Handling special characters includes addressing symbols and special characters. For example, text line “D-Dimer” requires special attention. Replacing the “-” with a blank space creates two different tokens, namely “D” and “Dimer”. As such, using traditional text preprocessing, the entire context of “D-Dimer” is lost. By addressing special characters, the context of the terms is maintained. Removing unwanted text from the event set hierarchy includes removing text that is present in all event set hierarchy data. Specifically, there are core event sets that are present in all event set hierarchy data. Since the core event sets do not add any new information between datasets, the core event sets are removed from the data. Custom preprocessing includes attending to consumer specific text, such as synonyms, abbreviations, and shorthand. The custom preprocessing may consult the synonyms, abbreviations, and shorthand 118 stored in the data repository 102 to provide expansions for various consumer specific synonyms, abbreviations, and shorthand.

In one or more embodiments, the vector embedding model 124 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GLOVE), Large Language Models (LLM), and BioWordVec fastText.

Each of these word embedding techniques includes salient features. The TF-IDF model is designed to give more weight to the words that are very specific to certain documents but less weight to the words that are more general and occur across most documents. The Word2Vec model represents words in the form of dense vectors by capturing syntactic (grammar) and semantic (meaning) relationships. Given a large enough dataset, the Word2Vec model provides strong estimates about a word's meaning based on its frequency of occurrence in the text. The GLOVE model is an unsupervised learning model that can be used to obtain dense word vectors like the Word2Vec model. The GLOVE model first creates a large word-context, co-occurrence matrix consisting of pairs (word, context). Each element in this matrix represents how often a word or a sequence of words occurs within the context and then applies matrix factorization to approximate this matrix. The BioWordVec fastText model is 200-dimensional word embeddings trained on PubMed and MIMIC-III data and is the extension of the original BioWord Vec that provides fastText word embeddings trained using PubMed and MeSH. A subword embedding model used by the BioWord Vec fastText model better handles Out of Vocabulary (OOV) tokens and improves the quality of the word embeddings.

In one or more embodiments, the word embedding technique includes Self-Alignment Pretraining for Biomedical Entity Representations (SAPBERT). The SAPBERT model leverages the Unified Medical Language System (UMLS), a comprehensive resource in the biomedical field. UMLS incorporates a vast collection of biomedical concepts and synonyms from various controlled vocabularies, like MeSH, SNOMED-CT, RxNorm, Gene Ontology, and OMIM. Use of these sources of data greatly enhances the model's understanding of medical terminology and relationships. SAPBERT model provides contextual embeddings, meaning that the model can understand the meaning of words and phrases in context. Context is crucial for understanding complex medical texts and making accurate predictions in healthcare applications. The SAPBERT model can accurately capture fine-grained semantic relationships and heterogeneous naming in the biomedical domain compared to other variants of BERT. The ability of SAPBERT to handle out-of-vocabulary (OOV) terms, misspelled words, and rare medical terms provides a significant advantage over other models.

A training data for the SAPBERT model consists of triplets (xₐ, xₚ, xₙ), where xₐ is the anchor entity, xₚ is the positive pair with xₐ, and xₙ is a negative pair with xₐ. λ is a pre-set margin. The SAPBERT model selects triplets that violate the condition:

$${‖ f ( {x}_{a} ) - f ( {x}_{b} ) ‖}_{2} < {‖ ( f ( {x}_{a} ) - f ( {x}_{n} ) ‖}_{2} + λ .$$

The equation represents that the distance between the anchor-positive pair should be less than the distance between the anchor-negative pair with some margin λ. This will ensure that samples are restricted to hard triplets. In other words, hard triplets consist of pairs where the distance of the anchor positive pair is more than the distance of the anchor-negative pair. For example, a hard triplet pair is (left nostril, left nare, right nostril). The embeddings generated by traditional BERT models for ‘left nostril’ and ‘right nostril’ are highly similar. During training, SAPBERT pushes apart the embedding of the anchor point from the negative point and brings the embedding of the anchor point closer to the positive point. SAPBERT uses Multi-Similarity (MS) loss that leverages similarities among and between positive and negative pairs to re-weight the importance of the samples.

In one or more embodiments, the similarity score calculator 126 calculates a similarity between vector embeddings for standard codes and vector embeddings for unmapped proprietary codes. The similarity score calculator 126 may include the Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the inverted file, Hierarchical Navigable Small World (HNSW), and product quantization. HNSW is an algorithm for efficient similarity searches in high-dimensional spaces. These indexing techniques help speed up nearest-neighbor searches in high-dimensional spaces. In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. Integration with libraries and frameworks may lead to significant improvements in the speed and scalability of the similarity search operations. As an open-source library, FAISS is available for developers and researchers to use, modify, and contribute to development of FAISS.

In one or more embodiments, standard code selector 128 provides recommendations for an unmapped proprietary code. The standard code selector 128 presents candidate mapped and unmapped standard codes to the user interface 106 based on the similarity values 114 provided by the similarity score calculator 126. The standard code selector 128 may present an “N” number of candidate standard codes ranked by the similarity values between the vector embeddings of the candidate standard codes and the vector embedding of the target unmapped proprietary code. Alternatively, the standard code selector 128 may present candidate standard codes having a similarity measure with the unmapped proprietary code above a threshold.

In some embodiments, the standard code selector 128 provides recommendations of one or more candidate unmapped proprietary codes for each standard code. The candidate unmapped proprietary codes may be presented in any of the same manners as described above with respect to the candidate standard codes.

In an embodiment, the mapping engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, user interface 106 refers to hardware and/or software configured to facilitate communications between a user and mapping engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, user interface 106 is specified in one or more other languages, such as Java, C, or C++.

### 3. Fine-Tuning System Architecture

FIG. 1B illustrates a fine-tuning system 130 in accordance with one or more embodiments. As illustrated in FIG. 1B, the fine-tuning system 130 is a component of or operates in combination with the mapping system 100. The fine-tuning system 130 includes a fine-tuning engine 132 for fine-tuning a pre-trained vector embedding model 134 to generate a fine-tuned vector embedding model 136. The fine-tuning system 130 may include more or fewer components than the components illustrated in FIG. 1B and may utilize the components illustrated in FIG. 1A. The components illustrated in FIG. 1B may be local to or remote from each other. The components illustrated in FIG. 1B may be implemented in software and/or hardware. Each component may be distributed over multiple applications and/or machines. Multiple components may be combined into one application and/or machine. Operations described with respect to one component may instead be performed by another component.

In one or more embodiments, the fine-tuning engine 132 includes a database 138, a selection module 140, a dataset generator 142, and a training module 144. Fine-tuning engine 132 operates to fine-tune the pre-trained vector embedding model 136 to generate the fine-tuned vector embedding model 136.

In one or more embodiments, database 138 may be populated with candidate standard codes 146, selected candidate standard codes 148, aggregated datasets 150, and training sets 152. Although shown in database 138, these components may also, or instead, be found in the data repository 102 (FIG. 1A) of mapping system 100.

In one or more embodiments, the candidate standard codes 146 are standard codes that are available for selection to be included in a training set for fine-tuning the pre-trained vector embedding model 134. The candidate standard codes 146 include standard codes that have been mapped to proprietary codes. Multiple proprietary codes may be mapped to a standard code. The candidate standard codes 146 may be maintained by healthcare data vendors, e.g., Cerner, Epic, 3M Healthcare, standards organizations and open access databases, e.g., LOINC, SNOMED-CT, RxNorm, fast healthcare interoperability resources APIs, e.g., LOINC FHIR, SNOMED-CT FHIR, manual mapping solutions, e.g., via terminologist and internal experts, open-source tools and community contributions, e.g., (OHDSI, OMOP), and health information exchanges (HIE).

In one or more embodiments, the selected candidate standard codes 148 refer to the candidate standard codes 146 that have been selected to be included in a training set for fine-tuning the pre-trained vector embedding model 134. The selected candidate standard codes 148 may be selected based on the candidate code meeting a threshold criteria.

In one or more embodiments, the aggregated datasets 150 are datasets associated with the selected candidate standard codes 148 and datasets associated with the proprietary codes that are mapped to the respective selected candidate standard codes 148. An aggregated dataset representing a selected candidate standard code may include text from one or more attributes of the selected candidate standard code. Similarly, an aggregated dataset representing a proprietary code that is mapped to a selected candidate standard code may include text from one or more attributes of the proprietary code. Machine learning may be used to determine the datasets that should be included in the aggregated datasets for the selected candidate standard codes and the corresponding proprietary codes.

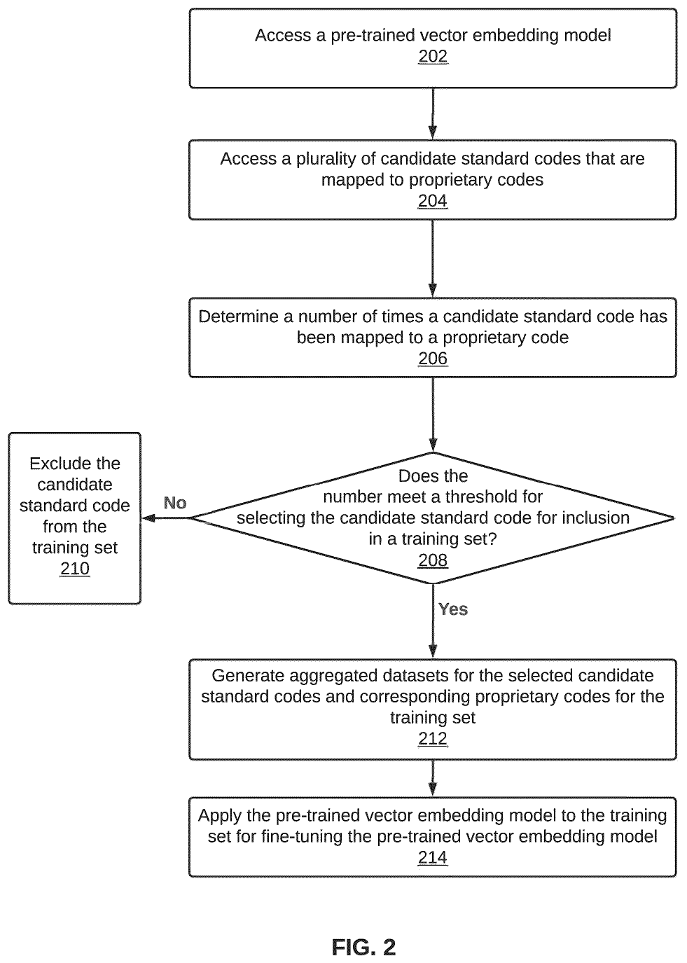

In one or more embodiments, the training sets 152 are training datasets used to fine-tune the pre-trained vector embedding model 134. A training dataset associated with a selected candidate standard code may include a label, e.g., an identifier corresponding to the selected candidate standard code, an aggregated dataset representing the selected candidate standard code, and an aggregated dataset representing a proprietary code that is mapped to the selected candidate standard code. The identifier may include an alpha-numeric sequence. The aggregated datasets may include datasets for one or more attributes of the selected candidate standard code. Example training datasets of a training set for use in fine-tuning a pre-trained vector embedding model are shown in FIG. 3.

In one or more embodiments, the selection module 140 refers to hardware and/or software configured to perform operations described herein for selecting candidate standard codes to be included in a training set for use in fine-tuning a pre-trained vector embedding model. Various techniques, e.g., uncertainty sampling, diversity sampling, active learning, hard example mining, similarity-based selection, domain-specific selection, may be used by selection module 140 to determine the candidate codes to select.

In one or more embodiments, the selection module 140 uses a number of times that a candidate standard code is mapped to a proprietary code to determine if the candidate standard code should be selected for use in a training set. The selection module 140 selects candidate standard codes that meet and/or exceed a threshold number of times the respective candidate standard code is mapped to a proprietary code. The threshold number may be two, three, or more times. Candidate standard codes whose mapping is limited to one or two proprietary codes may be considered outliers and may be excluded from the training set.

In one or more embodiments, the dataset generator 142 refers to hardware and/or software configured to perform operations described herein for generating training datasets for use in fine-tuning a pre-trained vector embedding model. Dataset generator 142 may generate training datasets that include aggregated datasets for selected candidate standard codes and aggregated datasets for the proprietary codes mapped to the selected candidate standard codes. Dataset generator 142 uses various techniques to determine what datasets of the selected candidate standard codes and what datasets of the proprietary codes to use in generating aggregated datasets for the selected candidate standard codes and the proprietary codes mapped to the respective selected candidate standard code.

In one or more embodiments, the dataset generator 142 uses a vector embedding model to generate vector embeddings for datasets associated with different attributes for a standard code. Using similarity measures, e.g., cosine similarity, the dataset generator 142 determines sets of attributes that meet a threshold measure and includes the datasets for those attributes in an aggregated dataset representing the selected candidate standard code.

In one or more embodiments, training module 144 refers to hardware and/or software configured to perform operations described herein for fine-tuning the pre-trained vector embedding model 134. Training module 144 uses learned weights and embeddings from a pre-trained vector embedding model and adapts the weights and embeddings to the new task by continuing the training process on a new training set. During training, training module 144 may update the model's parameters iteratively using backpropagation and gradient descent.

In one or more embodiments, the training module 144 uses online-batch-based hard triplets mining with a substantial batch size to enhance training efficiency. Online batch-based hard triplet mining is an approach that focuses on selecting the most informative triplets, e.g., anchor, positive, and negative samples, during training to enhance model performance and training efficiency. Triplet loss is used to learn embeddings by ensuring that an anchor sample is closer to a positive sample (of the same class) than to a negative sample (of a different class) by a certain margin. Since not all triplets are equally useful for training, hard triplet mining focuses on selecting triplets that are challenging for the model. Hard positives are positive samples that are far from the anchor, making the task of reducing the distance challenging. Hard negatives are negative samples that are close to the anchor, making the task of increasing the distance challenging. Instead of pre-defining hard triplets before training, online mining dynamically selects hard triplets within each training batch during the training process. This strategy ensures that the most challenging and informative samples are consistently used, enhancing the training process. Using a larger batch size increases the pool of available samples, allowing the mining process to find more diverse and genuinely hard triplets within each batch, leading to more effective training. Batch-wise online triplet mining introduces a form of regularization due to the random selection of samples within each mini-batch.

In one or more embodiments, training module 144 controls the configuration of fine-tuning hyperparameters. Hyperparameters that may be selected or adjusted for fine-tuning of a pre-trained vector embedding model include learning rate, number of epochs, optimizer, maximum length, and loss function. Learning rate is how much the model's weights, with respect to a gradient, are changed during training. Learning rates may be in the range of 2e⁻⁵ to 5e⁻⁵. A low learning rate may result in slower and more stable training, while a high learning rate may speed up training at the risk of overshooting the optimal solution. Number of epochs is the number of times the entire training dataset passes through the model. For fine-tuning tasks, the number of epochs may be in the range of three to five. Number of epochs may vary depending on dataset size and task complexity. More epochs allow the model to learn better; however, too many epochs can lead to overfitting, i.e., where the model performs well on training data but poorly on unseen data.

An optimizer is an algorithm that adjusts the weights of the neural network based on gradients. Common optimizers include Adam/AdamW and Stochastic Gradient Descent. Optimizers impact the speed and stability of training. Maximum length is the maximum number of tokens considered for each input text. Maximum lengths may include 128, 256, or 512 tokens. Increasing max length allows the model to process longer texts and requires more memory. Shorter max lengths speed up training by truncating long inputs; this can impact model performance if important information is lost. Loss function measures the difference between the model's predictions and the actual target values. Loss functions include, for example, cross-entropy loss, mean squared error (MSE), contrastive loss, or triplet loss. Loss functions impact how the model is penalized for making errors and shapes the model's training behavior.

Additional hyperparameters that may be selected or adjusted for fine-tuning of pre-trained vector embedding model may include automatic mixed precision (AMP), aggregation mode, miner margin, pairwise, and training batch size. AMP uses both 16-bit and 32-bit floating-point types to speed up training and reduce memory consumption. AMP may lead to faster training and lower memory usage. Aggregation mode is a method used to combine token embeddings into a single representation for a sequence or text. Aggregation modes include, for example, CLS, Mean, and Mean All Tokens. CLS uses a CLS token to represent the entire input sequence. Mean takes the mean of all token embeddings. Mean All Tokens is similar to Mean; however, Mean All Tokens may include all tokens, including padding tokens. Aggregation mode determines how the model generates a representation for a sequence. Miner margin defines the minimum distance between the positive and negative pairs for the model to consider a triplet loss effective. Miner margin impacts the difficulty of negative samples used in training. A higher margin forces the model to generate embeddings that are more distinctly separated, potentially improving similarity scoring performance. In pairwise training mode, the model learns from pairs of inputs (positive and negative examples). Training batch size is the number of samples processed before the model's weights are updated. Training batch sizes may include 8, 16, and/or 32 samples. Memory limitations may limit training batch size. A larger training batch size results in more stable gradients and requires more memory. Smaller batch sizes may introduce more noise into the learning process and can be beneficial when memory is limited.

In one or more embodiments, the pre-trained vector embedding model 134 is a machine learning model that has been trained on a large corpus of data to convert words, sentences, or other types of input into dense, fixed-size vectors in a continuous vector space, e.g., BERT, Word2Vec. The pre-trained vector embedding model 134 has been trained on a large general-purpose or domain-specific dataset, so the model may be directly used for downstream tasks, like classification, clustering, or similarity scoring, without the need for extensive training. The pre-trained vector embedding model is loaded with pre-trained weights of the pre-trained vector embedding model 134. The pre-trained weights may be stored in a serialized format, e.g., TensorFlow checkpoints or PyTorch state dictionaries. Parameters, such as a number of transformer layers, attention heads, hidden units, and vocabulary size, may be specified for the pre-trained vector embedding model 134.

In one or more embodiments, the fine-tuned vector embedding model 136 is a model that starts with a pre-trained vector embedding model and is further trained on a specific task or domain-specific data to improve the performance of the model for that particular task, e.g., similarity. Fine-tuning involves taking the learned weights and embeddings from the pre-trained vector embedding model and adapting them to the new task by continuing the training process on a new training dataset.

### 4. Generating Datasets for Fine-Tuning Pre-Trained Vector Embedding Models

FIG. 2 illustrates an example set of operations for generating a training set for fine-tuning a pre-trained vector embedding model in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

One or more embodiments access a pre-trained vector embedding model (Operation 202). Pre-trained vector embedding models, e.g., SAPBERT and BIOBERT, may be accessed from various platforms and libraries, e.g., Hugging Face Model Hub, TensorFlow Hub, Gensim, spaCy, Sentence-Transformers, and OpenAI API. The pre-trained vector embedding model uses fixed model weights determined during pre-training.

One or more embodiments access a plurality of candidate standard codes that are mapped to proprietary codes (Operation 204). A candidate standard code may have been mapped to one or more proprietary codes. Mappings may be maintained by healthcare data vendors, Standards organizations, healthcare institutions, or other interested parties. Mappings may be confirmed by a terminologist or other subject matter expert (SME). Candidate standard codes may be maintained in a table form and may be stored as a text file, e.g., CSV, JSON.

One or more embodiments determine a number of times each candidate standard code has been mapped to a proprietary code (Operation 206). A counting algorithm or other mechanism may be used to count the number of times a standard code has been mapped to a proprietary code. A candidate standard code that has been mapped to multiple proprietary codes may provide better results than a candidate standard code that has been mapped to a single proprietary code. The greater the number of proprietary codes that are mapped to a candidate standard code, the more relevant that candidate standard code may be to fine-tuning the pre-trained vector embedding model.

In one or more embodiments, a database query using SQL is used to count the occurrences of a standard code. The database query groups the mappings by standard code and counts how many times each standard code is mapped to a proprietary code. When the candidate standard codes are in a CSV file or a DataFrame, Python or Pandas may be used to perform the counting.

One or more embodiments determine if the number of times a candidate standard codes has been mapped to a proprietary code meets a threshold number for selecting the candidate standard code to be included in a training set (Operation 208). A threshold number “M” for selecting the candidate standard code to be included in the training set may vary. The greater the number of sets of proprietary codes being mapped to candidate standard codes, the greater the threshold number “M”. When the mappings include fewer sets of proprietary codes, the threshold number may be lower. The threshold number “M” may range from two (2) to multiple times.

Various other sampling and selection methods may be used for selecting candidate standard codes to be included in a training set for fine-tuning the pretrained vector embedding model.

When the number of times a candidate standard code has been mapped to a proprietary code does not meet a threshold number, one or more embodiments exclude the candidate standard code from the training set (Operation 210). A candidate standard code with less than two mappings to a proprietary may be considered an outlier. Inclusion of outliers in the training set may compromise the fine-tuning of the pre-trained vector embedding model.

One or more embodiments generate aggregated datasets representing the selected candidate standard codes and the corresponding proprietary codes for the training set (Operation 212). The aggregated datasets may include datasets associated with attributes for the selected candidate standard codes and the respective proprietary codes. The datasets used to generate the aggregated datasets may vary between selected candidate standard codes and between proprietary codes of the same and different sets of proprietary codes. An aggregated dataset representing a first selected candidate standard code may include a dataset from a first attribute and a dataset from a second attribute. An aggregated dataset representing a second selected candidate standard code is restricted to using the data from the first attribute. Similarly, an aggregated dataset representing a first proprietary code mapped to a selected candidate standard code may use multiple datasets for an attribute, while an aggregated dataset representing a second proprietary code mapped to the selected candidate standard code may use a single dataset for the attribute.

In one or embodiments, the system uses a pre-trained vector embedding model to generate vector embeddings for datasets associated with attributes of the selected candidate standard code. Using a similarity measure, e.g., cosine similarity, the system determines the datasets to include in an aggregated dataset representing the selected candidate standard code. For example, a first attribute for the selected candidate standard codes may include a common name, and a second attribute may include related names. Vector embeddings for the related names may vary greatly from the vector embedding for the common name. The larger the variation between vector embeddings, the greater the noise generated from including the dataset. To eliminate noise in a vector embedding for a selected candidate standard code, related common names for the selected candidate standard code with a vector embedding that do not meet a threshold similarity value with a vector embedding for the common name of the selected candidate standard code may be excluded, i.e., not selected, from the aggregated dataset representing the selected candidate standard code.

A selected candidate standard code may have multiple related names. Vector embedding for datasets of one or more of the related names may be sufficiently similar to the vector embedding for the dataset of the common name, i.e., meets the threshold value, to include the datasets of the one or more related names in the aggregated dataset of the selected candidate standard code. Conversely, vector embedding for datasets of one or more of the related names may be sufficiently different to the vector embedding for the dataset of the common name, i.e., fails to meet the threshold value, to exclude the datasets of the one or more related names from the aggregated dataset representing the selected candidate standard code.

Selecting the attributes of the proprietary codes may be performed in a similar manner. As attributes between sets of proprietary codes may differ, the attributes used between different sets of proprietary codes may differ. In an example, an aggregated dataset representing a proprietary code mapped to a selected candidate standard code includes a dataset for a first attribute, e.g., code name, and a dataset for a second attribute, Event Set Hierarchy.

One or more embodiments apply a pre-trained vector embedding model to the training set for fine-tuning of the pre-trained vector embedding model (Operation 214). Fine-tuning the pre-trained vector embedding model includes adjusting the hyperparameter configuration of the model. Hyperparameters may include learning rate, batch size, number of epochs, optimizer, and loss function. The hyperparameters selected may vary based on the specific task and dataset. The batch size may be larger than standard batch sizes, e.g., 128, 256, to provide a diverse pool of samples.

One or more embodiments uses online, batch-based, hard triplet mining with substantial batch size to fine-tune the pre-trained vector embedding model. Initially, the model may process the entire batch to generate vector embeddings for each dataset. The system computes the pairwise distances, e.g., cosine similarity, between the vector embeddings within the batch. A distance matrix may be generated that shows how close or far each sample is from other samples in the batch. Using the distance matrix, the system may identify potential triplets. Triple formation includes the following: anchor (A), a sample from a specific class; positive (P), a sample of the same class as the anchor meant to be close in the embedding space; and negative (N), a sample from a different class intended to be far from the anchor in the embedding space. Hard triplet mining includes the following: i) selecting positive samples that are farthest from the anchor among positive samples in the batch, i.e., hard positives; ii) selecting negative samples that are closest to the anchor, i.e., hard negatives, and optionally; and iii) selecting triplets where the negative is closer to the anchor than the positive, i.e., semi-hard triplets. The triplet loss function is then applied to the distances. The goal of triplet loss is to minimize a distance between an anchor and positive while maximizing the distance between the anchor and negative by a specified margin. The gradients of the triplet loss may then be calculated with respect to the model's parameters. The model parameters may be updated using an optimizer to minimize loss.

In one or more embodiments, the system periodically evaluates the model on a validation set to track the performance of the model during fine-tuning and adjusts parameters if necessary. The model may be saved at regular intervals or checkpoints and implement early stopping to prevent overfitting when the performance of the model's validation stops improving.

In one or more embodiments, the pre-trained vector embedding model is fine-tuned by leveraging uncertainty sampling and concentrating on sentence pairs where the model is least confident. Using similarity scores for unlabeled pairs, high-uncertainty pairs were identified and labeled. Identifying and labeling the high-uncertainty pairs may be performed by an SME or terminologist. The high-uncertainty pairs may then be used to curate the training set used to fine-tune the pretrained vector embedding model. This iterative process helps focus resources on the most informative data points and learn from challenging examples; this enhances accuracy and robustness more efficiently than random sampling and ultimately improves text similarity predictions.

In one or more embodiments, adversarial training was utilized during the fine-tuning of the pre-trained vector embedding model to bolster robustness and effectiveness of the model in addressing a wide range of complex textual variations. Adversarial training may include augmenting the training dataset with perturbed examples intended to mislead the model, promoting the acquisition of more stable features, and decreasing sensitivity to minor variations in input texts. Enhanced generalization capabilities and heightened resilience against potential adversarial inputs across real-world applications may be achieved through optimization against these adversarial instances in conjunction with conventional supervised learning goals.

### 5. Example Dataset of a Training Set for Fine-Tuning Pre-Trained Vector Embedding Models

A detailed example is described below for purposes of clarity. Components and/or operations described below should be understood as one specific example that may not be applicable to certain embodiments. Accordingly, components and/or operations described below should not be construed as limiting the scope of any of the claims.

FIG. 3 illustrates a format for the training datasets used in a training set for fine-tuning a pre-trained vector embedding model. The training set includes a label, a textual representation of a standard code, and a textual representation of a proprietary code that has been mapped to the standard code. The label is identified as concept_id, the textual representation of the standard code is identified as entity_name_1, and the textual representation of the proprietary code that has been mapped to the standard code is identified as entity_name_2.

In the example, the standard codes are LOINC codes, and the proprietary codes are from Code Set 72. The Concept ID for the first training dataset is LOINC code 10333-3. The first entity, represented by the Long Common Name for LOINC code 10333-3, is “appearance of cerebral spinal fluid”. The second entity, represented by the Code Name for the Code Set 72 code mapped to LOINC code 10333-3, is “appear csf”. The Concept ID for the second training dataset is LOINC code 10998-3. The first entity, represented by the Long Common Name for LOINC code 10998-3, is “oxycodone presence in urine”. The second entity, represented by the Code Name for the Code Set 72 code mapped to LOINC code 10998-3, is “oxycodone u”.

Although the textual representations of the standard codes and the textual representations of the proprietary codes each include a dataset from a single attribute, e.g., Long Common Name and Code Name, respectively, the textual representations may include datasets from more than one attribute.

### 6. Example Parameters for Fine-Tuning Pre-Trained Vector Embedding Models

A detailed example is described below for purposes of clarity. Components and/or operations described below should be understood as one specific example that may not be applicable to certain embodiments. Accordingly, components and/or operations described below should not be construed as limiting the scope of any of the claims.

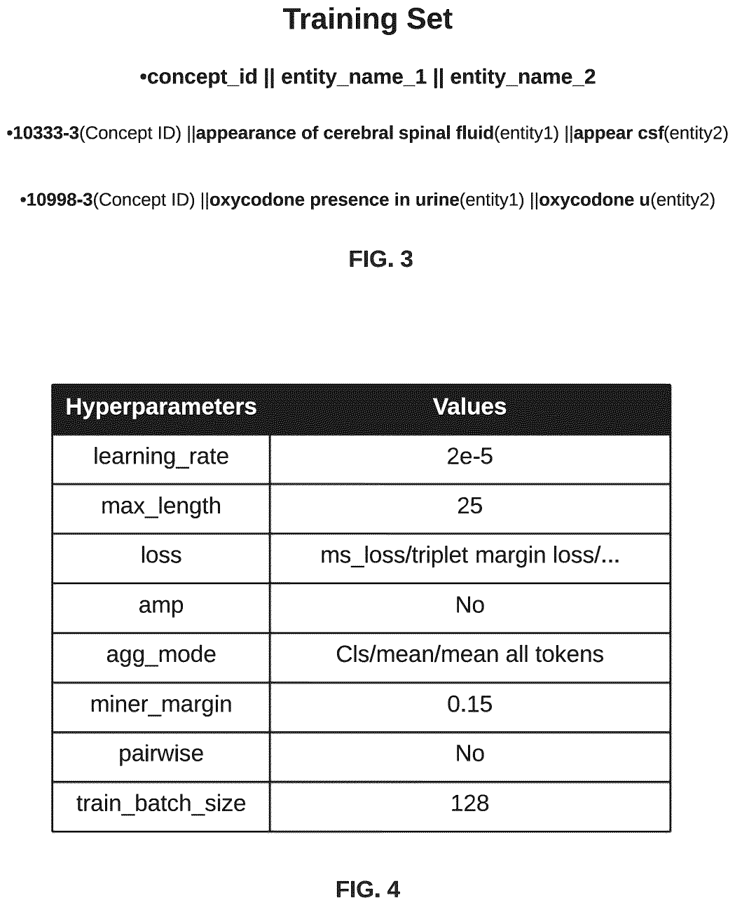

FIG. 4 illustrates an example of hyperparameters that may be adjusted for fine-tuning a pretrained vector embedding model. The hyperparameters include the following: learning rate, maximum length, loss, AMP, aggregation mode, miner margin, pairwise, and training batch size. In the example, the learning rate is set to 2e⁻⁵, maximum length is set to 25, the miner margin is set to 0.15, and the training batch size is set to 128. Both AMP and pairwise are set to No. By setting pairwise to No, triplet loss or contrastive loss are likely not used, and the model may instead use a standard classification loss, e.g., cross-entropy or other single-instance-based loss functions. By disabling AMP, the model will train using full 32-bit precision throughout.

### 7. Performance Comparison of Pre-Trained Vector Embedding Model and Fine-Tuned Vector Embedding Model

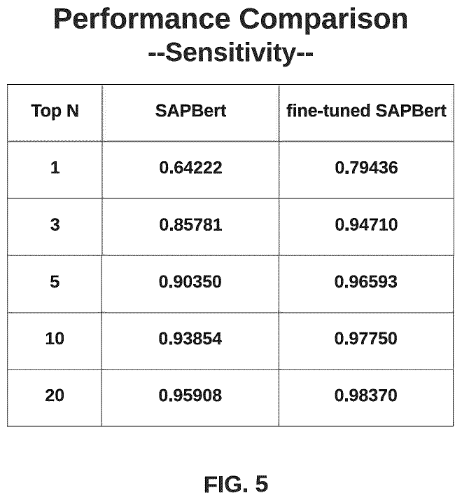

FIG. 5 is a chart illustrating performance of a fine-tuned SAPBERT model compared with baseline SAPBERT model. The top 5,000 LOINCs based on frequency were used to fine-tune the SAPBERT model. Sensitivity was used as an evaluation metric to gauge performance of the models. Sensitivity, also referred to as recall, is defined as a fraction of ‘relevant retrieved documents’ among ‘relevant documents in database’, as shown in the following equation.

$$recall = \frac{\vert \{ relevant document \} ∩ \{ retrieved documents \} \vert}{\{ relevant documents \}} .$$

Relevant documents are the documents that are truly relevant to the query or classification task. Retrieved documents are the documents that the system has retrieved or classified as relevant. “∩” denotes the overlap between the set of relevant documents and the set of retrieved documents, i.e., the true positives—the correctly identified relevant items.

The top 20 LOINIC codes were recommended for each client's proprietary code. The comparison of the sensitivity metric between the base SAPBERT model and the fine-tuned SAPBERT model for generating the top 1, 3, 5, 10 and 20 LOINC codes for each proprietary code is shown in FIG. 5. For Top1 matches, an improvement of ˜25% (0.64→0.79) is observed in the fine-tuned SAPBERT model over the base SAPBERT model.

### 8. Practical Application; Improvements & Advantages

Fine-tuning vector embedding models provides significant improvements in performance, accuracy, and applicability across various domain-specific tasks. By adjusting the model's parameters to better suit the nuances of specialized data, organizations may achieve more accurate and reliable outcomes in applications including search, recommendation, classification, and translation.

In one or more embodiments, fine-tuning improves the accuracy of matching proprietary codes to standard medical codes, thereby reducing manual reconciliation work. Fine-tuning embeddings may enhance text classification tasks by improving the contextual understanding of text inputs. Fine-tuning embeddings can improve cross-lingual tasks, including translating domain-specific content where general models may struggle.

In one or more embodiments, fine-tuning embeddings on domain-specific data allows models to capture nuances, jargon, and context better than general pre-trained models, improving task performance. By adjusting weights and refining embeddings for specific tasks, fine-tuned models can achieve higher accuracy in predictions, matching, or retrieval, leading to more reliable outcomes. Fine-tuning enables models to adapt to specific domains (e.g., finance, healthcare, legal), where language use differs significantly from general language models, improving their applicability. Fine-tuning on smaller, task-specific datasets can leverage pre-trained knowledge, reducing the need for extensive labeled data compared to training a model from scratch. Fine-tuning allows models to better understand domain-specific terms or rare words that are not well represented in general models. Models fine-tuned on domain-specific data often learn to handle noise or incomplete information more effectively, enhancing their real-world usability.

In one or more embodiments, fine-tuning allows tailoring of the embeddings specifically for a use case, improving performance on tasks like similarity scoring, classification, and/or retrieval. Fine-tuning is generally faster and less resource-intensive than training models from scratch, for it builds upon pre-trained knowledge. Fine-tuned models can generalize better within a specific domain, reducing errors when dealing with unseen but relevant examples. Leveraging existing pre-trained models allows for effective transfer learning, where general language understanding is transferred to a specialized context with minimal effort. Fine-tuning helps to avoid overfitting to a specific dataset by using a large, general pre-trained model as a base and making minor adjustments. Fine-tuned models can be easily updated or further refined with new data, allowing them to stay relevant as the domain evolves. Metrics, such as precision, recall, and F1-score, often improve with fine-tuning, leading to better overall model performance on evaluation benchmarks.

### 9. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

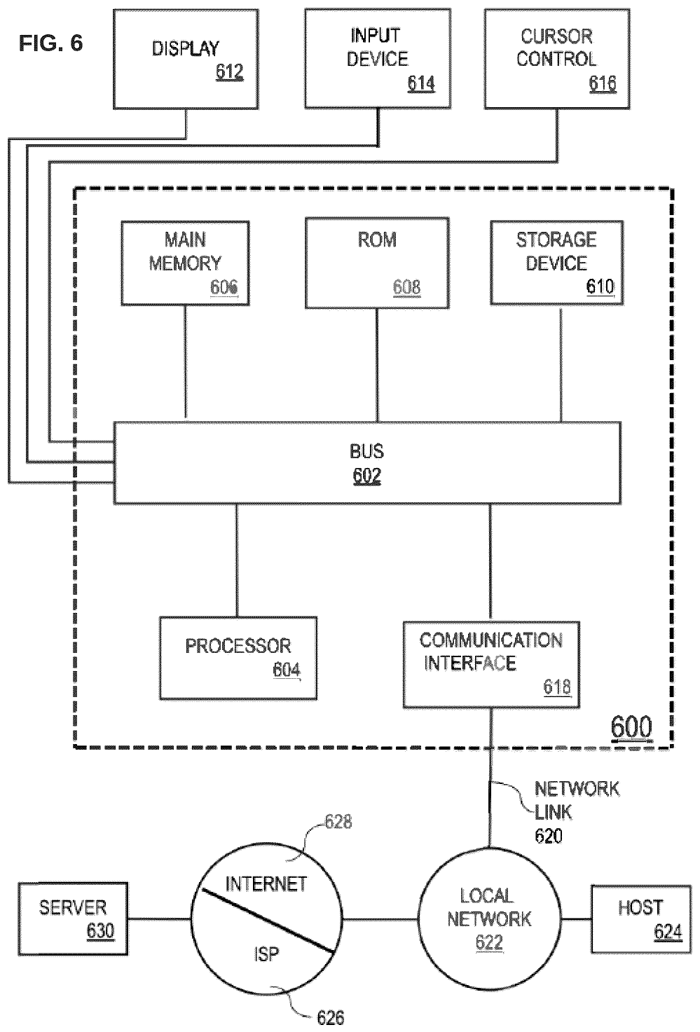

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 10. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a pre-trained vector embedding model; and
fine-tuning the pre-trained vector embedding model to generate a fine-tuned vector embedding model at least by:
accessing a candidate set of standard codes that have been mapped to proprietary codes;
determining a first number of times a first standard code has been mapped to a proprietary code;
responsive to the first number meeting a threshold number for use in fine-tuning the pre-trained vector embedding model, selecting the first standard code to be included in a training set for fine-tuning the pre-trained vector embedding model;
determining a second number of times a second standard code has been mapped to a proprietary code;
responsive to the second number not meeting the threshold number for use in fine-tuning, refraining from selecting the second standard code for to be included in a training set for fine-tuning the pre-trained vector embedding model; and
applying the pre-trained vector embedding model to the training set to generate the fine-tuned vector embedding model.

2. The one or more non-transitory computer readable media of claim 1, wherein fine-tuning the pre-trained vector embedding model further comprises:
generating a first aggregated dataset corresponding to the first standard code at least by:
generating a first vector embedding for a first dataset of the first standard code;
generating second vector embedding for a second dataset of the first standard code;
computing a first similarity measure for the first vector embedding and the second vector embedding; and
responsive to the first similarity measure meeting a threshold measure, selecting the second dataset to be included in the first aggregated dataset for the first standard code.

3. The one or more non-transitory computer readable media of claim 2, wherein generating the first aggregated dataset further comprises:
generating a third vector embedding for a third dataset of the first standard code;
computing a second similarity measure for the first vector embedding and the third vector embedding; and
responsive to the second similarity measure not meeting the threshold measure, refraining from selecting the third dataset to be included in the first aggregated dataset for the first standard code.

4. The one or more non-transitory computer readable media of claim 3, wherein fine-tuning the pre-trained vector embedding model further comprises:
generating a second aggregated dataset corresponding to one or more datasets of a first proprietary code that has been mapped to the first standard code,
wherein a first training dataset of the training set comprises:
a) an identifier corresponding to the first standard code,
b) the first aggregated dataset corresponding to the first standard code, and
c) the second aggregated dataset corresponding to the first proprietary code that has been mapped to the first standard code.

5. The one or more non-transitory computer readable media of claim 4, wherein fine-tuning the pre-trained vector embedding model further comprises:
generating a third aggregated dataset corresponding to one or more datasets of a second proprietary code mapped to the first standard code,
wherein a second training dataset of the training set comprises:
a) the identifier corresponding to the first standard code,
b) the first aggregated dataset corresponding to the first standard code, and
c) the third aggregated dataset corresponding to the second proprietary code that has been mapped to the first standard code.

6. The one or more non-transitory computer readable media of claim 4, wherein fine-tuning the pre-trained vector embedding model further comprises:
pre-processing the first aggregated dataset corresponding to the first standard code and the second aggregated datasets corresponding to the first proprietary code that has been mapped to the first standard code at least by:
a. converting text data into lowercase,
b. retaining numeric tokens,
c. handling special characters,
d. removing unwanted text from event set hierarchy, and
e. custom reprocessing for synonyms, abbreviations, and short hands.

7. The one or more non-transitory computer readable media of claim 1, wherein the pre-trained vector embedding model is Self-Alignment Pretraining for Biomedical Entity Representations (SAPBERT).

8. A method comprising:
accessing a pre-trained vector embedding model; and
fine-tuning the pre-trained vector embedding model to generate a fine-tuned vector embedding model at least by:
accessing a candidate set of standard codes that have been mapped to proprietary codes;
determining a first number of times a first standard code has been mapped to a proprietary code;
responsive to the first number meeting a threshold number for use in fine-tuning the pre-trained vector embedding model, selecting the first standard code for to be included in a training set for fine-tuning the pre-trained vector embedding model;
determining a second number of times a second standard code has been mapped to a proprietary code;
responsive to the second number not meeting the threshold number for use in fine-tuning, refraining from selecting the second standard code for to be included in a training set for fine-tuning the pre-trained vector embedding model; and
applying the pre-trained vector embedding model to the training set to generate the fine-tuned vector embedding model,
wherein the method is performed by at least one device including a hardware processor.

9. The method of claim 8, wherein fine-tuning the pre-trained vector embedding model further comprises:
generating a first aggregated dataset corresponding to the first standard code at least by:
generating a first vector embedding for a first dataset of the first standard code;
generating second vector embedding for a second dataset of the first standard code;
computing a first similarity measure for the first vector embedding and the second vector embedding; and
responsive to the first similarity measure meeting a threshold measure, selecting the second dataset to be included in the first aggregated dataset for the first standard code.

10. The method of claim 9, wherein generating the first aggregated dataset further comprises:
generating a third vector embedding for a third dataset of the first standard code;
computing a second similarity measure for the first vector embedding and the third vector embedding; and
responsive to the second similarity measure not meeting the threshold measure, refraining from selecting the third dataset to be included in the first aggregated dataset for the first standard code.

11. The method of claim 10, wherein fine-tuning the pre-trained vector embedding model further comprises:
generating a second aggregated dataset corresponding to one or more datasets of a first proprietary code that has been mapped to the first standard code,
wherein a first training dataset of the training set comprises:
d) an identifier corresponding to the first standard code,
e) the first aggregated dataset corresponding to the first standard code, and
f) the second aggregated dataset corresponding to the first proprietary code that has been mapped to the first standard code.

12. The method of claim 11, wherein fine-tuning the pre-trained vector embedding model further comprises:
generating a third aggregated dataset corresponding to one or more datasets of a second proprietary code mapped to the first standard code,
wherein a second training dataset of the training set comprises:
d) the identifier corresponding to the first standard code,
e) the first aggregated dataset corresponding to the first standard code, and
f) the third aggregated dataset corresponding to the second proprietary code that has been mapped to the first standard code.

13. The method of claim 11, wherein fine-tuning the pre-trained vector embedding model further comprises:
pre-processing the first aggregated dataset corresponding to the first standard code and the second aggregated datasets corresponding to the first proprietary code that has been mapped to the first standard code at least by:
f. converting text data into lowercase,
g. retaining numeric tokens,
h. handling special characters,
i. removing unwanted text from event set hierarchy, and
j. custom reprocessing for synonyms, abbreviations, and short hands.

14. The method of claim 8, wherein the pre-trained vector embedding model is Self-Alignment Pretraining for Biomedical Entity Representations (SAPBERT).

15. A system comprising:
at least one device including a hardware processor;
the system being configured to perform operations comprising:
accessing a pre-trained vector embedding model; and
fine-tuning the pre-trained vector embedding model to generate a fine-tuned vector embedding model at least by:
accessing a candidate set of standard codes that have been mapped to proprietary codes;
determining a first number of times a first standard code has been mapped to a proprietary code;
responsive to the first number meeting a threshold number for use in fine-tuning the pre-trained vector embedding model, selecting the first standard code to be included in a training set for fine-tuning the pre-trained vector embedding model;
determining a second number of times a second standard code has been mapped to a proprietary code;
responsive to the second number not meeting the threshold number for use in fine-tuning, refraining from selecting the second standard code to be included in a training set for fine-tuning the pre-trained vector embedding model; and
applying the pre-trained vector embedding model to the training set to generate the fine-tuned vector embedding model.

16. The system of claim 15, wherein fine-tuning the pre-trained vector embedding model further comprises:
generating a first aggregated dataset corresponding to the first standard code at least by:
generating a first vector embedding for a first dataset of the first standard code;
generating second vector embedding for a second dataset of the first standard code;
computing a first similarity measure for the first vector embedding and the second vector embedding; and
responsive to the first similarity measure meeting a threshold measure, selecting the second dataset to be included in the first aggregated dataset for the first standard code.

17. The system of claim 16, wherein generating the first aggregated dataset further comprises:
generating a third vector embedding for a third dataset of the first standard code;
computing a second similarity measure for the first vector embedding and the third vector embedding; and
responsive to the second similarity measure not meeting the threshold measure, refraining from selecting the third dataset to be included in the first aggregated dataset for the first standard code.

18. The system of claim 17, wherein fine-tuning the pre-trained vector embedding model further comprises:
generating a second aggregated dataset corresponding to one or more datasets of a first proprietary code that has been mapped to the first standard code,
wherein a first training dataset of the training set comprises:
g) an identifier corresponding to the first standard code,
h) the first aggregated dataset corresponding to the first standard code, and
i) the second aggregated dataset corresponding to the first proprietary code that has been mapped to the first standard code.

19. The system of claim 18, wherein fine-tuning the pre-trained vector embedding model further comprises:
generating a third aggregated dataset corresponding to one or more datasets of a second proprietary code mapped to the first standard code,
wherein a second training dataset of the training set comprises:
g) the identifier corresponding to the first standard code,
h) the first aggregated dataset corresponding to the first standard code, and
i) the third aggregated dataset corresponding to the second proprietary code that has been mapped to the first standard code.

20. The system of claim 15, wherein the pre-trained vector embedding model is Self-Alignment Pretraining for Biomedical Entity Representations (SAPBERT).
